(19) United States
(12) Patent Application Publication
(10) Pub. No.: US 2025/0235202 A1
(43) Pub. Date: Jul. 24, 2025

(54) LOCKING FERRULE DESIGNS FOR PERFORMING TENSIONABLE KNOTLESS SURGICAL PROCEDURES

(71) Applicant: ARTHREX, INC., Naples, FL (US)

(72) Inventors: Andrew Christian PETRY, Naples, FL (US); Peter J. DREYFUSS, Naples, FL (US); Asheesh BEDI, Glenview, IL (US)

(21) Appl. No.: 18/420,832

(22) Filed: Jan. 24, 2024

Related U.S. Application Data
(60) Provisional application No. 63/622,962, filed on Jan. 19, 2024.

(51) Int. Cl. A61B 17/04; A61B 17/06

(52) U.S. Cl. CPC A61B 17/0487; A61B 17/06166; A61B 2017/0417; A61B 2017/0445; A61B 2017/0456; A61B 2017/0462; A61B 2017/0496

(57) ABSTRACT

Systems and methods are provided for performing tensionable knotless surgical procedures. A locking ferrule that includes a one-way locking mechanism may be utilized for tensioning and locking one or more strands of suture during the surgical procedure. The one-way locking mechanism may be established by one or more locking barbs of the locking ferrule.

### CROSS-REFERENCE TO RELATED APPLICATIONS

This application claims the benefit of U.S. Provisional Application No. 63/622,962, which was filed on Jan. 19, 2024 and is incorporated hereby by reference in its entirety.

### BACKGROUND

This disclosure relates to the field of surgery, and more particularly to various locking ferrules that are capable of locking suture when performing tensionable knotless surgical procedures.

Repetitive trauma to a joint, such as a knee, ankle, hip, elbow, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to systems and methods for performing tensionable knotless surgical procedures, such as tissue repairs or reconstructions, for example. A locking ferrule that includes a one-way locking mechanism may be utilized during the tensionable knotless repairs for tensioning and locking one or more strands of suture.

An exemplary locking ferrule for performing a knotless tissue repair may include, inter alia, a body, a cannulation formed through the body, a plurality of locking barbs arranged within the cannulation and configured to establish a one-way locking mechanism for locking a suture that is shuttled through the cannulation, and a cut-out formed in the body.

An exemplary locking suture assembly may include, inter alia, a suture including a lumen, and a locking ferrule received within the lumen and including a one-way locking mechanism for locking a suture strand relative to the suture.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a locking ferrule that can be used for performing various tensionable knotless surgical procedures.

FIG. 2 is a top view of the locking ferrule of FIG. 1.

FIG. 3 is a cross-sectional view through section 3-3 of FIG. 2.

FIG. 4 is a cross-sectional view through section 4-4 of FIG. 1.

FIG. 5 schematically illustrates locking a suture relative to the locking ferrule of FIGS. 1-4.

FIG. 6 schematically illustrates a surgical method for performing a tissue repair.

FIG. 7 illustrates another exemplary locking ferrule that can be used for performing various tensionable knotless surgical procedures.

FIG. 8 is a cross-sectional view through section 8-8 of FIG. 7.

FIG. 9 illustrates an exemplary locking suture assembly.

FIG. 10 illustrates another exemplary locking suture assembly.

FIG. 11 illustrates another exemplary locking ferrule.

### DETAILED DESCRIPTION

This disclosure relates to systems and methods for performing tensionable knotless surgical procedures. A locking ferrule that includes a one-way locking mechanism may be utilized during the tensionable knotless repairs for tensioning and locking one or more strands of suture. These and other features of this disclosure are described in further detail below.

An exemplary locking ferrule for performing a knotless tissue repair may include, inter alia, a body, a cannulation formed through the body, a plurality of locking barbs arranged within the cannulation and configured to establish a one-way locking mechanism for locking a suture that is shuttled through the cannulation, and a cut-out formed in the body.

In any further embodiment, the body includes a cap portion that includes a top surface, a bottom surface, and a side wall that extends between the top surface and the bottom surface.

In any further embodiment, a barrel portion protrudes from the bottom surface of the cap portion.

In any further embodiment, the cut-out extends radially into the side wall and opens through both the top surface and the bottom surface of the cap portion.

In any further embodiment, the cut-out is configured to allow the suture to pass through the cap portion prior to entering the cannulation at a distal end of the barrel portion.

In any further embodiment, the body includes a barrel portion that includes an interior wall and an outer diameter wall.

In any further embodiment, the cut-out extends radially into the outer diameter wall.

In any further embodiment, the cut-out is configured to allow the suture to pass around a proximal end of the barrel portion prior to entering the cannulation at a distal end of the barrel portion.

In any further embodiment, each of the plurality of locking barbs is angled in a direction toward a cap portion of the body.

In any further embodiment, each of the plurality of locking barbs protrudes inwardly from an interior wall that circumscribes the cannulation.

In any further embodiment, the plurality of locking barbs are arranged in at least a first row and a second row within the cannulation.

In any further embodiment, a first portion of the plurality of locking barbs of the first row are staggered relative to a second portion of the plurality of locking barbs of the second row.

In any further embodiment, each of the plurality of locking barbs includes a pointed tip.

In any further embodiment, the suture includes a varying thickness.

In any further embodiment, the suture includes at least one tapered region where the suture transitions between a thickened section and a thinned section.

An exemplary locking suture assembly may include, inter alia, a suture including a lumen, and a locking ferrule received within the lumen and including a one-way locking mechanism for locking a suture strand relative to the suture.

In any further embodiment, the suture strand is an integral portion of the suture.

In any further embodiment, the suture strand is a separate and distinct suture from the suture.

In any further embodiment, the suture is a single-lumen suture.

In any further embodiment, the suture is a multi-lumen suture.

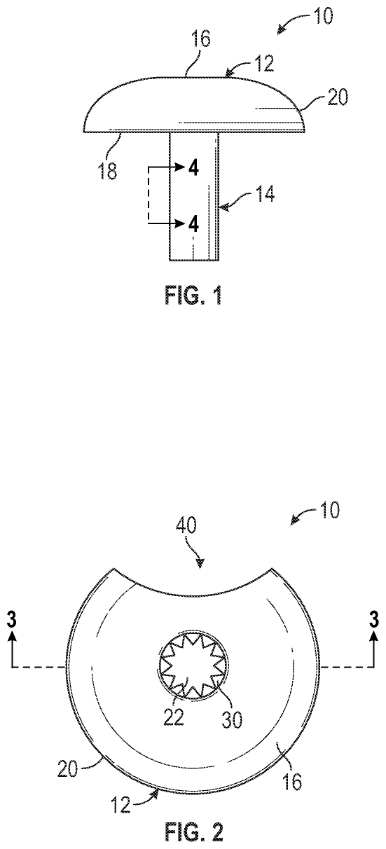

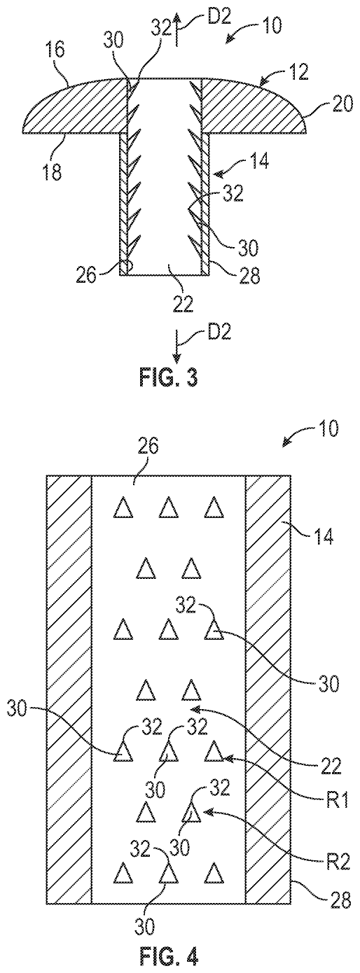

FIGS. 1-5 illustrate an exemplary locking ferrule 10 that can be used when performing various tensionable knotless surgical procedures. For example, the locking ferrule 10 could be utilized during surgical methods for attaching tissue (e.g., ligament, tendon, graft, etc.) to bone or for repairing any other type of tissue defect. The locking ferrule 10 could be used in conjunction with a variety of orthopedic surgical repairs or reconstructions, including but not limited to rotator cuff repairs, Achilles tendon repairs, patellar tendon repairs, and biceps tendon repairs, among many others.

The locking ferrule 10 is an exemplary suture locking device that can include a cap portion 12 and a barrel portion 14 that protrudes from the cap portion 12. The cap portion 12 and the barrel portion 14 may be formed together as part of a unitary body structure of the locking ferrule 10. In an embodiment, the cap portion 12 and the barrel portion 14 are arranged to provide a top hat-like appearance of the locking ferrule 10. However, the specific size, shape, and material make-up of the locking ferrule 10 are not intended to limit this disclosure.

The cap portion 12 of the locking ferrule 10 may include a top surface 16, a bottom surface 18, and a side wall 20 that extends between the top surface 16 and the bottom surface 18. The bottom surface 18 may include a slightly concave curvature for conforming to the contour of a bone. The barrel portion 14 may protrude outwardly from the bottom surface 18 of the cap portion 12. As further detailed below, the locking ferrule 10 may be positioned relative to a bone such that the barrel portion 14 is accommodated within a socket or tunnel formed in the bone and the cap portion 12 is seated relative to the cortex of the bone. In the seated position, the top surface 16 of the cap portion 12 faces away from the bone and the bottom surface 18 faces toward the bone.

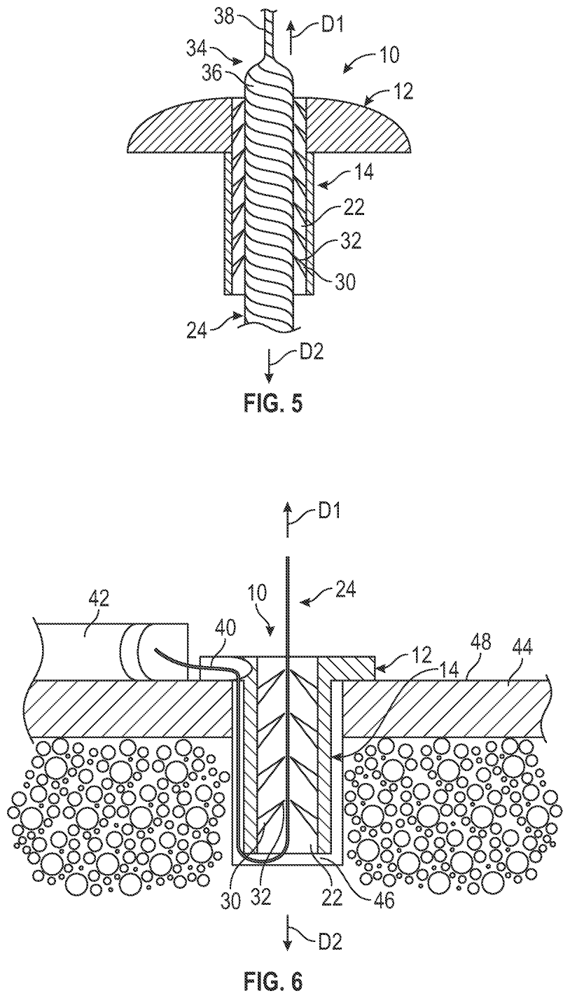

A cannulation 22 may extend through the cap portion 12 and the barrel portion 14. The cannulation 22 may establish an internal passageway for accommodating one or more strands of suture (see, for example, suture 24 of FIG. 5) or other structures (e.g., wires, shafts, etc.).

The cannulation 22 may be circumscribed by an interior wall 26 of the locking ferrule 10. The interior wall 26 may be established by both the cap portion 12 and the barrel portion 14. The cannulation 22 may be straight or may taper in a direction toward a distal tip of the barrel portion 14. An outer diameter wall 28 of the barrel portion 14 may be smooth or could alternatively include threads, barbs, or other features for facilitating bone fixation.

A plurality of locking barbs 30 may protrude inwardly from the interior wall 26. The locking barbs 30 may therefore occupy at least a portion of the open space of the cannulation 22. In an embodiment, the locking barbs 30 are integrally formed (e.g., molded) features of the body of the locking ferrule 10. The locking barbs 30 may be provided along an entire length of the cannulation 22 or at only select portions thereof. The locking barbs 30 may be either rigid or flexible structures.

The locking barbs 30 may be arranged in multiple rows along the length of the cannulation 22. For example, the locking barbs 30 may be arranged in a least a first row R1 and a second row R2 (see FIG. 4). In an embodiment, the locking barbs 30 of the second row R2 are staggered relative to the locking barbs 30 of the first row R1 (see FIG. 4).

Each locking barb 30 may include a sharp or pointed tip 32, and, in this implementation, each locking barb 30 may be angled in a direction toward the cap portion 12. The locking barbs 30 may therefore establish a one-way locking mechanism that permits one or more sutures 24 to pass through the cannulation 22 in a first direction D1 while preventing the suture(s) 24 from being tensioned or otherwise moved in a second direction D2. The staggered relationship of the rows of locking barbs 30 can maximize engagement with the suture 24 once it is passed through the cannulation 22.

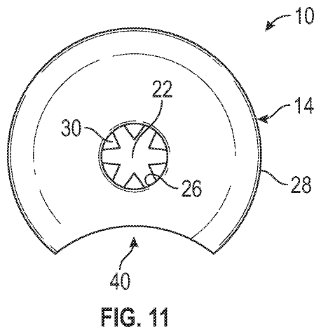

One or more scallops or cut-outs 40 may be formed in the side wall 20 of the cap portion 12. Each cut-out 40 may extend radially into the side wall 20 and may open through both the top surface 16 and the bottom surface 18 of the cap portion 12. In another implementation, the locking ferrule 10 may exclude the cap portion 12, and therefore the cut-out 40 may extend radially into the outer diameter wall 28 of the barrel portion 14 (see, e.g., FIG. 11). In either case, the cut-outs 40 can provide clearance for passing suture 24 into and back out of the locking ferrule 10 even after the locking ferrule 10 has been implanted into a bone hole or some other tissue structure. The suture 24 may therefore be passed through the cap portion 12 or a proximal end of the barrel portion 14 prior to entering the cannulation 22 at a distal end of the barrel portion 14.

Referring now primarily to FIG. 5, with continued reference to FIGS. 1-4, one or more sutures 24 may be passed through the cannulation 22 of the locking ferrule 10. The suture 24 may be FiberWire®, FiberTape®, or any other suitable suture product. FiberWire® and FiberTape® are suture products marketed and sold by Arthrex, Inc. However, other suture products could be utilized within the scope of this disclosure. The size and type of suture utilized in conjunction with the locking ferrule 10 are not intended to limit this disclosure

The suture 24 may include a varying thickness. The suture 24 may therefore include one or more tapered regions 34 where the suture 24 transitions between a thickened section 36 and a thinned section 38. The thinned sections 38 can facilitate passing the suture 24 through the cannulation 22, such as via a suitable suture loader, for example, and the thickened sections 36 can provide greater surface area for the locking barbs 30 to engage with in order to sufficiently lock the suture 24 relative to the locking ferrule 10.

In an embodiment, the thickened sections 36 of the suture 24 are about twice as thick as the thinned sections 38 of the suture 24. However, other ratios (e.g., 1.5:1, 3:1, etc.) between the relative thicknesses (e.g., outer diameters) of the thickened section 36 and the thinned section 38 are contemplated within the scope of this disclosure.

The thinned sections 38 of the suture 24 may be shuttled through the cannulation 22, and the suture 24 may then be tensioned in the first direction D1 to lock the thickened section 36 of the suture 24 relative to the locking ferrule 10. The locking barbs 30 prevent the suture 24 from backing up or otherwise moving in the second direction D2. The pointed tips 32 of the locking barbs 30 may interdigitate with the thickened section 36 of the suture 24 in order to lock the suture 24 and prevent it from moving in the second direction D2.

Although shown as locking a single unfolded suture 24 in the above implementations, the locking ferrule 10 could be configured to receive and lock a folded suture, multiple strands of suture, and/or sutures of varying shapes and sizes.

The locking ferrules 10 described above and shown in FIGS. 1-5 may be utilized to tension and knotlessly fixate one or more sutures 24 as part of various surgical methods. FIG. 6 schematically illustrates one such surgical method for attaching a tissue 42 (e.g., ligament, tendon, muscle, etc.) to a bone 44. The tissue 42 may have torn away from the bone 44 during vigorous exercise or sporting activities, for example. When such tears occur, reattachment is often necessary to repair the tissue defect.

The surgical method may include preparing a socket 46 in the bone 44. The socket 46 may be a preformed opening formed in the bone 44 that is sized for receiving the barrel portion 14 of the locking ferrule 10. The locking ferrule 10 may be implanted such that the barrel portion 14 is accommodated within the socket 46 and the cap portion 12 is seated relative to a cortex 48 of the bone 44.

The suture 24 may be passed through or around the tissue 42 and may then be shuttled through the cannulation 22 of the locking ferrule 10. The suture 24 may be passed through the cannulation 22 of the locking ferrule 10 either before or after inserting the barrel portion 14 of the locking ferrule 10 into the socket 46. If the barrel portion 14 has already bene inserted into the socket 46, the suture 24 may be passed through the cut-out 40 of the cap portion 12, down into the socket 46, and then back up through the cannulation 22 to a location outside of the bone 44. The suture 24 may then be tensioned in the direction D1 to knotlessly secure the tissue 42 at a desired position relative to the bone 44. Tensioning the suture 24 in the direction D1 allows the pointed tips 32 of the locking barbs 30 to interdigitate with the portion of the suture 24 accommodated within the cannulation 22, thereby locking the suture 24 in place and preventing it from sliding back in the second direction D2.

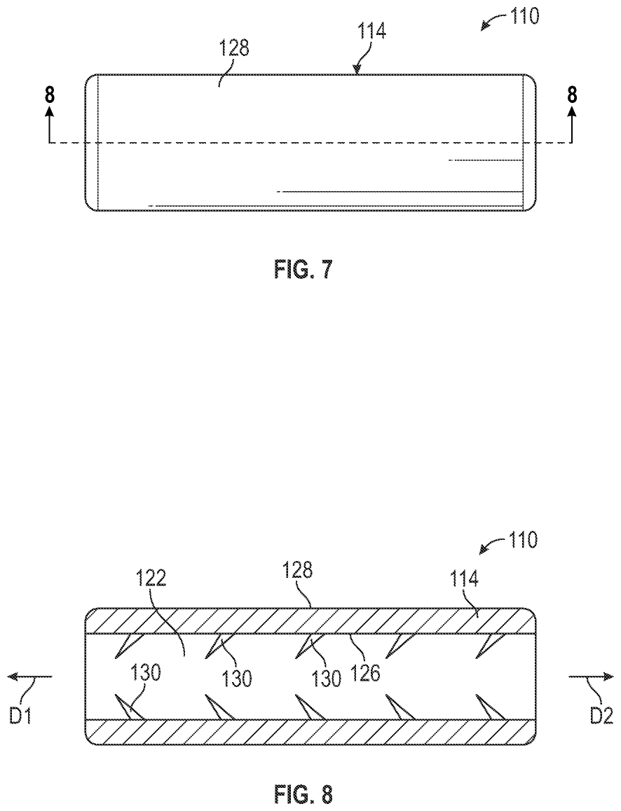

FIGS. 7 and 8 illustrate another exemplary locking ferrule 110 that can be used when performing various tensionable knotless surgical procedures. The locking ferrule 110 is similar to the locking ferrule 10 described above but includes only a barrel portion 114 without the integrated cap portion.

A cannulation 122 may extend through the barrel portion 114 and may establish an internal passageway for accommodating one or more strands of suture. The cannulation 122 may be circumscribed by an interior wall 126 of the locking ferrule 110. An outer diameter wall 128 of the barrel portion 114 may be smooth or could alternatively include threads, barbs, or other features for facilitating bone fixation.

A plurality of locking barbs 130 may protrude inwardly from the interior wall 126. The locking barbs 130 may therefore occupy at least a portion of the open space of the cannulation 122. The locking barbs 130 may establish a one-way locking mechanism that permits suture to pass through the cannulation 122 in a first direction D1 while preventing suture from being tensioned or otherwise moved in a second direction D2.

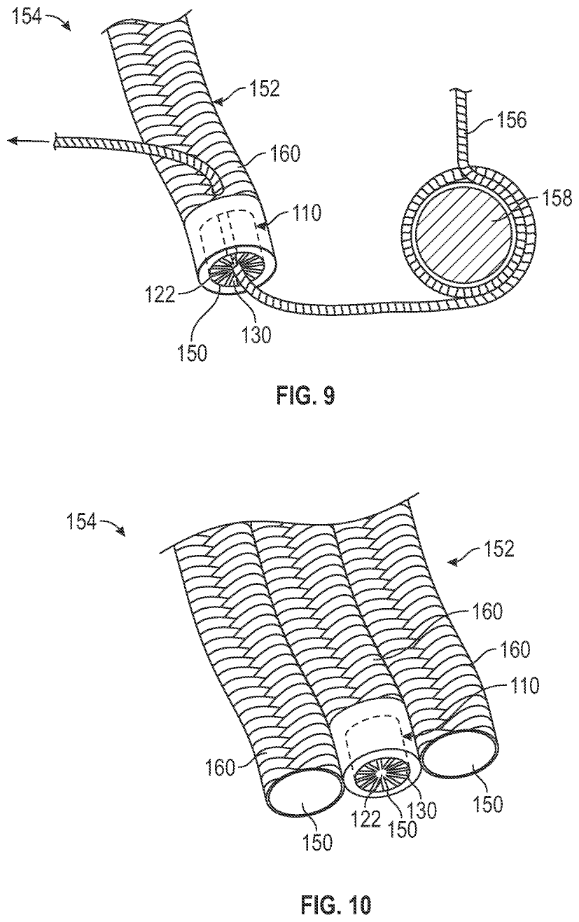

Referring now to FIG. 9, the locking ferrule 110 may be inserted into a lumen 150 of a suture 152 to establish a locking suture assembly 154. The suture 152 may be a single-lumen suture (see FIG. 9) or a multi-lumen suture (see FIG. 10).

A suture strand 156 may be passed through or around a tissue 158 (e.g., bone, ligament, tendon, muscle, etc.), then be shuttled through the cannulation 122 of the locking ferrule 110, and then be shuttled through an outer braided jacket 160 of the suture 152 to a location outside of the suture 152. The outer braider jacket 160 circumscribes the lumen 150. The suture strand 156 may be either an integral end portion of the suture 152 or a separate and distinct suture that is passed through the suture 152 to provide a suture-through-suture arrangement. Once shuttled through the locking ferrule 110, the suture strand 156 may be tensioned to lock the suture strand in place relative to the locking ferrule 110 and thereby knotlessly secure the tissue 158, such as when performing a cerclage technique or a side-to-side tissue closure, for example.

The suture locking devices of this disclosure may be utilized for performing various tensionable knotless surgical repairs. The suture locking devices provide for tensioning and retensioning suture(s) at various points of the repair, including subsequent to implantation of accompanying fixation devices, thus providing numerous advantages over prior tissue repair systems and techniques.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A locking ferrule for performing a knotless tissue repair, comprising:
a body;
a cannulation formed through the body;
a plurality of locking barbs arranged within the cannulation and configured to establish a one-way locking mechanism for locking a suture that is shuttled through the cannulation; and
a cut-out formed in the body.

2. The locking ferrule as recited in claim 1, wherein the body includes a cap portion that includes a top surface, a bottom surface, and a side wall that extends between the top surface and the bottom surface.

3. The locking ferrule as recited in claim 2, comprising a barrel portion that protrudes from the bottom surface of the cap portion.

4. The locking ferrule as recited in claim 3, wherein the cut-out extends radially into the side wall and opens through both the top surface and the bottom surface of the cap portion.

5. The locking ferrule as recited in claim 4, wherein the cut-out is configured to allow the suture to pass through the cap portion prior to entering the cannulation at a distal end of the barrel portion.

6. The locking ferrule as recited in claim 1, wherein the body includes a barrel portion that includes an interior wall and an outer diameter wall.

7. The locking ferrule as recited in claim 6, wherein the cut-out extends radially into the outer diameter wall.

8. The locking ferrule as recited in claim 7, wherein the cut-out is configured to allow the suture to pass around a proximal end of the barrel portion prior to entering the cannulation at a distal end of the barrel portion.

9. The locking ferrule as recited in claim 1, wherein each of the plurality of locking barbs is angled in a direction toward a cap portion of the body.

10. The locking ferrule as recited in claim 1, wherein each of the plurality of locking barbs protrudes inwardly from an interior wall that circumscribes the cannulation.

11. The locking ferrule as recited in claim 1, wherein the plurality of locking barbs are arranged in at least a first row and a second row within the cannulation.

12. The locking ferrule as recited in claim 11, wherein a first portion of the plurality of locking barbs of the first row are staggered relative to a second portion of the plurality of locking barbs of the second row.

13. The locking ferrule as recited in claim 1, wherein each of the plurality of locking barbs includes a pointed tip.

14. The locking ferrule as recited in claim 1, wherein the suture includes a varying thickness.

15. The locking ferrule as recited in claim 14, wherein the suture includes at least one tapered region where the suture transitions between a thickened section and a thinned section.

16. A locking suture assembly, comprising:
a suture including a lumen; and
a locking ferrule received within the lumen and including a one-way locking mechanism for locking a suture strand relative to the suture.

17. The locking suture assembly as recited in claim 16, wherein the suture strand is an integral portion of the suture.

18. The locking suture assembly as recited in claim 16, wherein the suture strand is a separate and distinct suture from the suture.

19. The locking suture assembly as recited in claim 16, wherein the suture is a single-lumen suture.

20. The locking suture assembly as recited in claim 16, wherein the suture is a multi-lumen suture.
